(19) United States
(12) Patent Application Publication
(10) Pub. No.: US 2005/0095287 A1
(43) Pub. Date: May 5, 2005

(54) Fosinopril composition

(76) Inventors: Amol Singh Matharu, Bedminster, NJ (US); Mahendra R. Patel, East Brunswick, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/915,507

(22) Filed: Aug. 10, 2004

Related U.S. Application Data
(60) Provisional application No. 60/515,331, filed on Oct. 29, 2003.

(51) Int. Cl. A61K 31/66; A61K 9/20

(52) U.S. Cl. 424/464; 514/102

(57) ABSTRACT

This disclosure relates to stable pharmaceutical formulations of the drug fosinopril sodium comprising a lubricant which is sodium stearate or a mixture composed of stearic acid esters, propylene glycol monoesters, distilled monoglycerides, sodium stearoyl lactylate, and silicon dioxide.

### BACKGROUND

The invention relates to stable pharmaceutical formulations of fosinopril sodium and to the use of specific lubricants in the formulation. Fosinopril sodium is marketed as a drug for controlling high blood pressure and for heart failure under the brandname MONOPRIL by Bristol Myers Squibb Company.

Fosinopril is disclosed in U.S. Pat. No. 4,337,201. U.S. Pat. No. 5,006,344 discloses that the shelf life and stability of fosinopril sodium formulated as tablets is increased when the lubricant employed is sodium stearyl fumarate or hydrogenated vegetable oil compared to tablets containing magnesium stearate as the lubricant. These patents are incorporated herein by reference. According to the Physician's Desk Reference, 56^{th} Edition, MONOPRIL brand fosinopril sodium tablets include sodium stearyl fumarate as the lubricant, in addition to lactose, microcrystalline cellulose, crospovidone, povidone, and the active ingredient, fosinopril sodium.

The present invention relates to the discovery that the shelf life and stability of fosinopril sodium formulated as tablets is also increased, compared to tablets containing magnesium stearate as the lubricant, when the lubricant employed is sodium stearate or the MYVATEX TL tablet lubricant, which is available from Eastman Fine Chemicals. In addition, the tablets obtained possess improved compression properties. MYVATEX TL is a mixture composed of stearic acid esters, propylene glycol monoesters, distilled monoglycerides, sodium stearoyl lactylate, and silicon dioxide. Sodium stearate is commonly used as a surfactant in toothpastes and gum and has not been used as a lubricant in a pharmaceutical formulation.

### DETAILED DESCRIPTION

The invention relates to a pharmaceutical tablet formulation which comprises a pharmaceutically effective amount of fosinopril sodium and a pharmaceutically acceptable carrier comprising a filler and a lubricant wherein the lubricant is selected from the group consisting of sodium stearate and the tablet lubricant MYVATEX TL.

Fosinopril sodium is marketed as 10 mg, 20 mg, and 40 mg tablets. However, pharmaceutically effective amounts within the meaning of the present invention include smaller and larger doses that provide a desirable physiological effect.

Fillers are well-known in the pharmaceutical formulation arts. Suitable fillers usable herein include any pharmaceutically acceptable filler such as lactose, microcrystalline cellulose, and combinations of lactose and microcrystalline cellulose. Lactose is a preferred filler.

Preferably, a pharmaceutical tablet formulation of the present invention comprises from 1 to about 25 percent by weight of fosinopril sodium, from about 25 to about 90 percent by weight of the filler, and from about 0.3 to about 10 percent by weight of the lubricant.

In one embodiment of the invention, the lubricant is sodium stearate.

In another embodiment of the invention, the lubricant is the mixture composed of stearic acid esters, propylene glycol monoesters, distilled monoglycerides, sodium stearoyl lactylate, and silicon dioxide.

The pharmaceutical tablet formulation of the invention preferably comprises from about 0.3 to about 5 percent by weight of the lubricant.

In addition to the active ingredient, the filler, and the lubricant, pharmaceutical tablet formulations of the invention can further comprise a binder and an optional disintegrant.

Suitable binders include povidone, i.e., 1-ethenyl-2-pyrrolidinone homopolymer, hydroxypropyl cellulose, and mixtures thereof.

It is not necessary to use a disintegrant in the formulations of the invention, as the requirement of the disintegrant is dictated by the solubility of the active drug. Fosinipril is highly soluble and hydroscopic. However, if desired, conventional pharmaceutical excipients can be used. Suitable disintegrants include microcrystalline cellulose, sodium carboxymethyl starch, cross-linked sodium carboxymethyl starch, crospovidone, i.e., 1-ethenyl-2-pyrrolidinone homopolymer, cross-linked sodium carboxymethylcellulose (which is available as AcDiSol or Croscarmellose Sodium), sodium starch glycolate, and mixtures thereof Some pharmaceutical excipients act both as a binder and as a disintegrant. For example, pre-gelatinzed starch can be employed as both disintegrant and binder.

Preferably, the binder, if present, is in an amount of from about 1 to 5 percent by weight of the formulation.

Preferably, the disintegrant, if present, is in an amount of from 0 to 10 percent by weight of the formulation.

The invention includes pharmaceutical tablet formulations which contain only fosinopril sodium as the active ingredient or a combination of fosinopril with a second active ingredient. Suitable second active ingredients include, for example, a diuretic, such as hydrochlorothiazide; a calcium channel blocker, such as amlodipine; an angiotensin II receptor antagonist, such as irbesartan; an alpha and/or beta andregenic blocking agent, such as carvedilol; or an HMG-CoA inhibitor, such pravastatin sodium. A preferred second active ingredient is hydrochlorothiazide which is present in amounts from 1 to 25 percent by weight of the formulation, preferably 12.5 percent.

Other ingredients commonly employed in pharmaceutical formulations can also be included herein, such as coloring agents or flavors. The tablets of the invention are prepared using standard tableting techniques.

The following examples are intended to further describe, but not limit, the present invention.

### EXAMPLE 1

COMPOSITION OF FOSINOPRIL SODIUM TABLETS

| Ingredient | % per unit | 10 mg | | 20 mg | | 40 mg | |
|---|---|---|---|---|---|---|---|
| Fosinopril Sodium | 12.500 | 10.00 | | 20.0 | | 40.0 | |
| Lactose Anhydrous, DT | 82.813 | 66.25 | | 132.5 | | 265.0 | |
| Povidone K-30 | 1.875 | 1.50 | | 3.0 | | 6.0 | |
| Purified Water* | None | q.s. | | q.s. | | q.s. | |
| Sodium Stearate | 2.813 | 2.25 | | 4.5 | | 9.0 | |
| TOTAL WEIGHT | 100% | 80 | mg | 160 | mg | 320 | mg |

*Used in the manufacturing process only. Does not appear in the final product.

Stability of Fosinopril Sodium tablets
at 40° C./75% RH
(100 count, Induction sealed HDPE bottles; with dessicant)

| | | |
|---|---|---|
| 10 mg | 20 mg | 40 mg |

% Assay
(% total degradation products)

| | | | |
|---|---|---|---|
| Initial | 98.3 | 96.9 | 98.6 |
| | (0.27) | (0.28) | (0.3) |
| 1 month | 99.9 | 99.1 | 97.7 |
| | (0.5) | (0.53) | (0.63) |
| 2 month | 98.5 | 98.9 | 95.9 |
| | (0.68) | (0.76) | (0.87) |
| 3 month | 99.4 | 97.7 | 96.7 |
| | (1.04) | (0.92) | (1.1) |

Stability of Fosinopril Sodium tablets
at RT (25° C./60% RH)
(100 count, Induction sealed HDPE bottles; with dessicant)

| | | |
|---|---|---|
| 10 mg | 20 mg | 40 mg |

% Assay
(% total degradation products)

| | | | |
|---|---|---|---|
| Initial | 98.3 | 96.9 | 98.6 |
| | (0.27) | (0.28) | (0.3) |
| 3 month | 99.4 | 97.7 | 96.7 |
| | (0.35) | (0.92) | (1.1) |
| 6 month | 98.3 | 96.0 | 96.8 |
| | (0.42) | (0.42) | (0.53) |
| 9 month | 99.5 | 95.3 | 98.0 |
| | (0.47) | (0.52) | (0.24) |
| 12 month | 99.8 | 96.7 | 97.3 |
| | (0.81) | (0.62) | (0.88) |

1. A pharmaceutical tablet formulation which comprises a pharmaceutically effective amount of fosinopril sodium and a lubricant wherein the lubricant is selected from the group consisting of sodium stearate and a mixture composed of stearic acid esters, propylene glycol monoesters, distilled monoglycerides, sodium stearoyl lactylate, and silicon dioxide.

2. A formulation of claim 1 further comprising a filler.

3. A formulation of claim 2 which comprises from 1 to about 25 percent by weight of fosinopril sodium, from about 25 to about 90 percent by weight of the filler, and from about 0.3 to about 10 percent by weight of the lubricant.

4. A formulation of claim 1 wherein the lubricant is sodium stearate.

5. A formulation of claim 3 wherein the lubricant is sodium stearate.

6. A formulation of claim 5 which comprises from about 0.3 to about 5 percent by weight sodium stearate.

7. A formulation of claim 7 which further comprises a binder or a disintegrant or both.

8. A formulation of claim 7 wherein the binder is present in an amount of from about 1 to 5 percent by weight.

9. A formulation of claim 1 wherein the lubricant is the mixture composed of stearic acid esters, propylene glycol monoesters, distilled monoglycerides, sodium stearoyl lactylate, and silicon dioxide.

10. A formulation of claim 3 wherein the lubricant is the mixture composed of stearic acid esters, propylene glycol monoesters, distilled monoglycerides, sodium stearoyl lactylate, and silicon dioxide.

11. A formulation of claim 10 which comprises from about 0.3 to about 5 percent by weight of the mixture composed of stearic acid esters, propylene glycol monoesters, distilled monoglycerides, sodium stearoyl lactylate, and silicon dioxide.

12. A formulation of claim 11 which further comprises a binder or a disintegrant or both.

13. A formulation of claim 12 wherein the binder is present in an amount of from about 1 to 5 percent by weight.

14. A formulation of claim 1 further comprising a second active ingredient wherein the., second active ingredient is selected from the group consisting of a diuretic, a calcium channel blocker, an angiotensin II receptor antagonist, an alpha and/or beta andregenic blocking agent, and an HMG-CoA inhibitor

15. A formulation of claim 14 wherein the second active ingredient is selected from the group consisting of hydrochlorothiazide, amlodipine, irbesartan, carvedilol, and pravastatin sodium.

16. A formulation of claim 15 further comprising a filler.

17. A formulation of claim 16 which comprises from 1 to about 25 percent by weight of fosinopril sodium, from 1 to about 25 percent by weight of hydrochlorothiazide, from about 25 to about 90 percent by weight of the filler, and from about 0.3 to about 10 percent by weight of the lubricant.

18. A formulation of claim 17 wherein the lubricant is sodium stearate.

19. A pharmaceutical tablet formulation comprising about 12.5 percent by weight fosinopril sodium, about 82.8 percent by weight anhydrous lactose, about 1.875 percent by weight povidone, and about 2.8 percent by weight sodium stearate.
